(19) United States
(12) Patent Application Publication
(10) Pub. No.: US 2022/0374795 A1
(43) Pub. Date: Nov. 24, 2022

(54) UTILITY DETERMINATION PREDICTIVE DATA ANALYSIS SOLUTIONS USING MAPPINGS ACROSS RISK DOMAINS AND EVALUATION DOMAINS

(71) Applicant: Optum, Inc., Minnetonka, MN (US)

(72) Inventors: Lun Yu, San Francisco, CA (US); Huzaifa Sial, Emeryville, CA (US); Pooja Rao, Millbrae, CA (US)

(21) Appl. No.: 17/468,057

(22) Filed: Sep. 7, 2021

Related U.S. Application Data
(60) Provisional application No. 63/190,405, filed on May 19, 2021.

(51) Int. Cl. G06Q 10/06; G06K 9/62

(52) U.S. Cl. CPC G06Q 10/0635; G06K 9/6272; G06K 9/6277

(57) ABSTRACT

Various embodiments of the present invention provide methods, apparatus, systems, computing devices, computing entities, and/or the like for performing predictive utility evaluation for a utility measure value given a defined demographic profile. Certain embodiments of the present invention utilize systems, methods, and computer program products that perform predictive utility evaluation for a utility measure value given a defined demographic profile by utilizing mappings across risk domains and evaluation domains to generate predicted utility scores based at least in part on utility distribution measures that are determined based at least in part on risk distribution measures, for example based at least in part on relationships between a predicted utility score and a distribution of predicted utility scores for a plurality of utility discretization categories.

### CROSS-REFERENCES TO RELATED APPLICATION(S)

The present application claims priority to the U.S. Provisional Application No. 63/190,405, filed on May 19, 2021, which is incorporated by reference herein in its entirety.

### BACKGROUND

Various embodiments of the present invention address technical challenges related to performing predictive data analysis and provide solutions to address the efficiency and reliability shortcomings of various existing predictive data analysis solutions.

### BRIEF SUMMARY

In general, various embodiments of the present invention provide methods, apparatus, systems, computing devices, computing entities, and/or the like for performing predictive utility evaluation for a utility measure value given a defined demographic profile. Certain embodiments of the present invention utilize systems, methods, and computer program products that perform predictive utility evaluation for a utility measure value given a defined demographic profile by utilizing mappings across risk domains and evaluation domains to generate predicted utility scores based at least in part on utility distribution measures that are determined based at least in part on risk distribution measures, for example based at least in part on relationships between a predicted utility score and a distribution of predicted utility scores for a plurality of utility discretization categories.

In accordance with one aspect, a method is provided. In one embodiment, the method comprises: identifying a plurality of utility discretization categories for a utility measure that is associated with the utility measure value, wherein: (i) each utility discretization category is associated with a risk distribution measure value for the defined demographic profile, and (ii) the plurality of utility discretization categories comprise an observed utility discretization category that describes a current utility measure value for the utility measure value; for each utility discretization category, determining a utility distribution measure value based at least in part on transforming the risk distribution measure value for the utility discretization category into a utility domain; determining the predicted utility score based at least in part on a relationship between the utility distribution measure value for the utility discretization category that is associated with observed utility discretization category and each utility distribution measure value for the plurality of utility discretization categories; and performing one or more prediction-based actions based at least in part on the predicted utility score.

In accordance with another aspect, a computer program product is provided. The computer program product may comprise at least one computer-readable storage medium having computer-readable program code portions stored therein, the computer-readable program code portions comprising executable portions configured to: identify a plurality of utility discretization categories for a utility measure that is associated with the utility measure value, wherein: (i) each utility discretization category is associated with a risk distribution measure value for the defined demographic profile, and (ii) the plurality of utility discretization categories comprise an observed utility discretization category that describes a current utility measure value for the utility measure value; for each utility discretization category, determine a utility distribution measure value based at least in part on transforming the risk distribution measure value for the utility discretization category into a utility domain; determine the predicted utility score based at least in part on a relationship between the utility distribution measure value for the utility discretization category that is associated with observed utility discretization category and each utility distribution measure value for the plurality of utility discretization categories; and perform one or more prediction-based actions based at least in part on the predicted utility score.

In accordance with yet another aspect, an apparatus comprising at least one processor and at least one memory including computer program code is provided. In one embodiment, the at least one memory and the computer program code may be configured to, with the processor, cause the apparatus to: identify a plurality of utility discretization categories for a utility measure that is associated with the utility measure value, wherein: (i) each utility discretization category is associated with a risk distribution measure value for the defined demographic profile, and (ii) the plurality of utility discretization categories comprise an observed utility discretization category that describes a current utility measure value for the utility measure value; for each utility discretization category, determine a utility distribution measure value based at least in part on transforming the risk distribution measure value for the utility discretization category into a utility domain; determine the predicted utility score based at least in part on a relationship between the utility distribution measure value for the utility discretization category that is associated with observed utility discretization category and each utility distribution measure value for the plurality of utility discretization categories; and perform one or more prediction-based actions based at least in part on the predicted utility score.

### BRIEF DESCRIPTION OF THE DRAWINGS

Having thus described the invention in general terms, reference will now be made to the accompanying drawings, which are not necessarily drawn to scale, and wherein:

FIG. 1 provides an exemplary overview of an architecture that can be used to practice embodiments of the present invention.

FIG. 2 provides an example predictive data analysis computing entity in accordance with some embodiments discussed herein.

FIG. 3 provides an example client computing entity in accordance with some embodiments discussed herein.

FIG. 4 is a flowchart diagram of an example process for predictive utility evaluation for a utility measure value given a defined demographic profile in accordance with some embodiments discussed herein.

FIG. 5 provides an operational example of a utility measure hierarchical scheme in accordance with some embodiments discussed herein.

FIG. 6 is a flowchart diagram of an example process for determining a utility distribution measure value for each utility discretization category of a plurality of utility discretization categories in accordance with some embodiments discussed herein.

FIG. 7 provides an operational example of determining a predicted utility score for each utility discretization category of a plurality of utility discretization categories in accordance with some embodiments discussed herein.

FIG. 8 is a flowchart diagram of an example process for performing one or more prediction-based actions based at least in part on a predicted utility score for a utility measure value in accordance with some embodiments discussed herein.

FIG. 9 is a flowchart diagram of an example process for determining an overall predicted utility score for a monitored entity in accordance with some embodiments discussed herein.

FIG. 10 provides an operational example of a prediction output user interface describing a relationship between a predicted utility score for a utility measure value and a distribution of predicted utility scores for the plurality of utility discretization categories in accordance with some embodiments discussed herein.

### DETAILED DESCRIPTION

Various embodiments of the present invention now will be described more fully hereinafter with reference to the accompanying drawings, in which some, but not all, embodiments of the inventions are shown. Indeed, these inventions may be embodied in many different forms and should not be construed as limited to the embodiments set forth herein; rather, these embodiments are provided so that this disclosure will satisfy applicable legal requirements. The term “or” is used herein in both the alternative and conjunctive sense, unless otherwise indicated. The terms “illustrative” and “exemplary” are used to be examples with no indication of quality level. Like numbers refer to like elements throughout. Moreover, while certain embodiments of the present invention are described with reference to predictive data analysis, one of ordinary skill in the art will recognize that the disclosed concepts can be used to perform other types of data analysis.

### I. Overview and Technical Improvements

Various embodiments of the present invention improve the efficiency of performing utility evaluation by introducing techniques to determine predicted utility scores by mapping values from a risk domain to a utility domain. The inventors have concluded that, in a lot of application contexts, risk scores are more readily available than utility scores. By generating predicted utility scores by mapping risk scores to utility scores, various embodiments of the present invention avoid the need for performing computationally intensive operations to generate utility measures from raw feature data. In this way, various embodiments of the present invention improve the computational efficiency of performing utility evaluation.

Moreover, various embodiments of the present invention improve the operational reliability of predicted utility scores generated using performing utility evaluation by generating predicted utility scores based at least in part on a relationship between a utility distribution measure value for the utility discretization category that is associated with an observed utility discretization category and each utility distribution measure value for a plurality of utility discretization categories. In some embodiments, the noted task may be performed based at least in part on the output of the equation

$$\frac{u - min}{max - min} ,$$

where min is the minimum value of the utility distribution values for the set of utility discretization categories and max is the maximum value of the utility distribution values for the set of utility discretization categories. In some embodiments, the operations of the described equation (and potentially other normalization equations) have the effect that, if u=max, the predicted utility score is one, and if u=min, the predicted utility score is zero, a technique which enhances the diversity of the predicted utility scores within an allowable range and does not “waste” any unused subranges of the allowable range. In this way, the noted embodiments of the present invention generate predicted utility scores that have a wider precision and thus are deemed more predictively informative and operationally reliable. Accordingly, by utilizing the described techniques, various embodiments of the present invention improve the operational reliability of predicted utility scores generated using performing utility evaluation.

### II. Definitions

The term “utility discretization category” may refer to a data construct that is configured to describe a designation for a set of utility measure values associated with a utility measure. In some embodiments, all of the potential utility measure values for a particular utility measures are divided into a set of disjoint utility discretization categories, such that each potential utility measure value is assigned to not more than and no less than one utility discretization category. For example, if a utility measure is a continuous value (e.g., a body mass index (BMI) measure), then a total range of the utility measure may be divided into a number of sub-ranges each corresponding to a utility discretization category. In one example, if a utility measure may have a continuous value in the range [0, 1], then the utility discretization categories for the utility measure may include a first utility discretization category for utility measure values that fall in the sub-range [0, 0.25), a second utility discretization category for utility measure values that fall in the sub-range [0.25, 0.5), a third utility discretization category for utility measure values that fall in the sub-range [0.5, 0.75), and a fourth utility discretization category for utility measure values that fall in the sub-range [0.75, 1]. As another example, if a utility measure is a discrete value (e.g., a number of weekly exercise sessions), then all potential discrete values for the utility measure may be divided into a number of “buckets” each corresponding to a utility discretization category. In one example, if a utility measure may have a discrete value from the set {0, 1, 2, 3, 4}, then the utility discretization categories for the utility measure may include a first utility discretization category for utility measure values that fall in the subset {0,1}, a third utility discretization category for utility measure values that fall in the subset {2, 3}, and a third utility discretization category for utility measure values that fall in the subset {4}.

The term “utility measure” may refer to a data construct that is configured to describe an aspect of an overall utility measurement with respect to an observed set of phenomena. For example, a set of utility measures may include a set of measurements/observations that relate to health state and/or health activities of a monitored individual. In some embodiments, a utility measure may be associated with a utility category and a utility sub-category. For example, a utility measure that describes an aspect of a health state and/or a health activity of a monitored individual may have a utility category that describes that the utility measure pertains to biometric observations/measurements, and a utility sub-category that describes that the utility measure pertains to a particular biometric observation/measurement, such as to a BMI biometric observation/measurement. Examples of utility categories include a biometrics utility category, a diet utility category, an exercise utility category, a sleep utility category, and a state of mind utility category. Examples of utility sub-categories for a biometrics utility category include a BMI utility sub-category, a dyslipidemia utility sub-category, a blood pressure utility sub-category, and/or the like. Examples of utility sub-categories for a nutrition utility category include a nutrition score utility sub-category, a calorie count utility sub-category, and/or the like. Examples of utility sub-categories for an exercise utility category include an aerobic exercise session count utility sub-category, an exercise session count utility sub-category, a weight-bearing exercise session count utility sub-category, and/or the like. Examples of utility sub-categories for a sleep utility category include a sleep hour magnitude utility sub-category, a sleep quality score utility sub-category, and/or the like. Examples of utility sub-categories for a state of mind utility category include a mood score utility sub-category, a social isolation score utility sub-category, a stress score utility sub-category, and/or the like. In some embodiments, a utility category describes a biometrics utility category and a utility sub-category for the utility category describes a particular biometric measurement. In some embodiments, a utility category describes an exercise activity utility category and a utility sub-category of the utility category describes a recorded count of performance of a particular exercise activity. In some embodiments, a utility measure value is a particular value of a utility measure that may be associated with a monitored entity (e.g., a monitored individual). For example, if a utility measure is associated with weekly exercise session count, then a utility measure value may describe a weekly exercise session count of 4 that may be associated with a particular monitored individual.

The term “defined demographic profile” may refer to a data construct that is configured to describe a combination of feature values corresponding for a combination of features associated with a set of monitored entities (e.g., a set of monitored individuals), such that the set of monitored entities may be divided into a number of sets comprising a subset that is associated with the defined demographic profile. An example of a defined demographic profile is a defined demographic profile associated women between 40-50 years of age. Another example of a defined demographic profile is a defined demographic profile associated with women between 40-50 years of age that live in the United States. A yet another example of a defined demographic profile is a defined demographic profile associated with women between 40-50 years of age that live in the United States and have diabetes. In some embodiments, each defined demographic profile is associated with a subset of a set of monitored entities that have the feature values characterizing the defined demographic profile. For example, with respect to the defined demographic profile associated with women between 40-50 years of age that live in the United States and have diabetes, the relevant subset may include all those monitored individuals that are women, that have 40-50 years of age, that live in the United States, and that have diabetes.

The term “risk distribution measure value” may refer to a data construct that is configured to describe a measure of statistical distribution of a risk measure across a defined demographic profile given a utility discretization category that is associated with the risk distribution measure. For example, consider a utility discretization category that describes performing three or four weekly exercises and a defined demographic profile of women between 40-50 years of age that live in the United States and have diabetes. In the noted example, the risk distribution measure value may describe a statistical distribution measure (e.g., a mean) of a risk measure (e.g., an episode risk group (ERG) measure) across all monitored individuals that correspond to the defined demographic profile (i.e., across all monitored individuals that are women between 40-50 years of age, live in the United States, and have diabetes). Thus, in the noted example, each utility measure such as a weekly exercise session count utility measure may be associated with n risk distribution measure values, where n is the number of utility discretization categories (e.g., buckets) associated with the utility measure. In some embodiments, to determine a risk distribution measure value for a defined demographic profile given a utility discretization category, the following operations may be performed: (i) identifying all of the monitored entities (e.g., all of the monitored individuals) that both satisfy the defined demographic profile (e.g., are associated with the required demographic features) and are associated with utility measure values that fall within the set of utility measures values associated with the utility discretization category (e.g., that have been recorded to have exercised the required number of times weekly); (ii) for each identified monitored entity, determining a relevant risk measure value (e.g., an ERG value); (iii) compute a measure of statistical distribution of each relevant risk measure value for an identified monitored entity; and (iv) determine the risk distribution measure value based at least in part on the measure of statistical distribution. While various embodiments of the present invention describe using mean values to determine risk distribution measure values, a person of ordinary skill in the relevant technology will recognize that other statistical distribution values, such as other centroid statistical distribution values, can be used to determine risk distribution measures values. Other examples of statistical distribution values that can be used to determine risk distribution measures values include median values, weighted mean values, mode values, and/or the like. In some embodiments, each risk distribution measure value describes a centroid statistical distribution measure for all risk measure values (e.g., ERG values) for monitored entities (e.g., monitored individuals) that are associated with the defined demographic profile and the utility discretization category for the risk distribution measure value.

The term “utility distribution measure value” may refer to a data construct that is configured to describe the output of a transformation of a risk distribution measure value to a utility domain. In some embodiments, a risk distribution measure value is associated with a risk domain (e.g., the ERG risk domain), which may describe a scheme for mapping a set of monitored entities into a set of numbers/values (e.g., a set of risk distribution measure values) such that a monitored entity deemed to be associated with greater risk is associated with a higher number/value in the risk domain. Therefore, if monitored entity m₁ is deemed to be in a riskier condition than the monitored entity m₂ (e.g., m₁ is deemed to have a BMI that is further away from a healthy BMI range than the BMI for m₂), then the mapped number/value for m₁ in the risk domain is higher than the mapped number/value for m₂ in the risk domain. On the other hand, a utility distribution measure value is associated with a utility domain, which may describe a scheme for mapping a set of monitored entities into a set of numbers/values (e.g., a set of utility distribution measure values) such that a monitored entity deemed to be associated with greater risk is associated with a lower number/value in the utility domain. Therefore, if monitored entity m₁ is deemed to be in a riskier condition than the monitored entity m₂ (e.g., m₁ is deemed to have a BMI that is further away from a healthy BMI range than the BMI for m₂), then the mapped number/value for m₁ in the utility domain is lower than the mapped number/value for m₂ in the utility domain. Thus, in some embodiments, generating a utility distribution measure value for a utility discretization category comprises identifying the risk distribution measure value for the utility discretization category and then mapping the risk distribution measure value into the utility domain in order to generate the utility distribution measure value for the utility discretization category. This may include, in some embodiments, inversing or negating the risk distribution measure value for the particular utility discretization category. For example, in some embodiments, given a utility discretization category that is associated with the risk distribution measure value r, the utility discretization measure value for the utility discretization category is determined based at least in part on the output of 1/r or r⁻¹.

The term “predicted utility score” may refer to a data construct that is configured to describe, for a utility measure value (e.g., of a monitored entity such as a monitored individual) that is associated with a particular observed utility discretization category, a relationship between the utility distribution measure value for the observed utility discretization category and each utility distribution measure value for the plurality of utility discretization categories. In some embodiments, each utility measure value is associated with a particular utility discretization category, and each utility discretization category is associated with a utility distribution measure. For example, a particular utility measure that is associated with weekly exercise session counts may be associated with a first utility discretization category having a utility distribution measure of u₁ that applies to monitored entities having {0, 1} weekly exercise session counts, a second utility discretization category having a utility distribution measure u₂ that applies to monitored entities having {2, 3, 4} weekly exercise session counts, and a third utility discretization category having a utility distribution measure u₃ that applies to monitored individuals having more than 4 weekly exercise session counts. In the noted example, the predicted utility score for a monitored individual having three weekly exercise session counts may be determined based at least in part on a relationship between u₂ and {u₁, u₂, u₃}. For example, the predicted utility score may be determined based at least in part on normalizing (e.g., min-max normalization of) each of u₁, u₂, and u₃. As another example, the predicted utility score may be determined based at least in part on: the utility distribution measure value for the observed utility discretization category (e.g., u₂), a minimal predicted utility score for the plurality of utility discretization categories (a minimum value of u₁, u₂, and u₃), and a maximal predicted utility score for the plurality of utility discretization categories (a maximum value of u₁, u₂, and u₃). In some embodiments, given a utility distribution measure value of u for a particular monitored individual that is associated with a particular utility discretization category of a set of utility discretization categories, the predicted utility score for the particular monitored individual and/or for the particular utility discretization category may be determined based at least in part on the output of the equation

$$\frac{u - min}{max - min} ,$$

where min is the minimum value of the utility distribution values for the set of utility discretization categories and max is the maximum value of the utility distribution values for the set of utility discretization categories. In some embodiments, the operations of the described equation (and potentially other normalization equations) have the effect that, if u=max, the predicted utility score is one, and if u=min, the predicted utility score is zero, a technique which enhances the diversity of the predicted utility scores within an allowable range and does not “waste” any unused subranges of the allowable range.

### III. Computer Program Products, Methods, and Computing Entities

Embodiments of the present invention may be implemented in various ways, including as computer program products that comprise articles of manufacture. Such computer program products may include one or more software components including, for example, software objects, methods, data structures, or the like. A software component may be coded in any of a variety of programming languages. An illustrative programming language may be a lower-level programming language such as an assembly language associated with a particular hardware architecture and/or operating system platform. A software component comprising assembly language instructions may require conversion into executable machine code by an assembler prior to execution by the hardware architecture and/or platform. Another example programming language may be a higher-level programming language that may be portable across multiple architectures. A software component comprising higher-level programming language instructions may require conversion to an intermediate representation by an interpreter or a compiler prior to execution.

Other examples of programming languages include, but are not limited to, a macro language, a shell or command language, a job control language, a script language, a database query or search language, and/or a report writing language. In one or more example embodiments, a software component comprising instructions in one of the foregoing examples of programming languages may be executed directly by an operating system or other software component without having to be first transformed into another form. A software component may be stored as a file or other data storage construct. Software components of a similar type or functionally related may be stored together such as, for example, in a particular directory, folder, or library. Software components may be static (e.g., pre-established or fixed) or dynamic (e.g., created or modified at the time of execution).

A computer program product may include a non-transitory computer-readable storage medium storing applications, programs, program modules, scripts, source code, program code, object code, byte code, compiled code, interpreted code, machine code, executable instructions, and/or the like (also referred to herein as executable instructions, instructions for execution, computer program products, program code, and/or similar terms used herein interchangeably). Such non-transitory computer-readable storage media include all computer-readable media (including volatile and non-volatile media).

In one embodiment, a non-volatile computer-readable storage medium may include a floppy disk, flexible disk, hard disk, solid-state storage (SSS) (e.g., a solid state drive (SSD), solid state card (SSC), solid state module (SSM), enterprise flash drive, magnetic tape, or any other non-transitory magnetic medium, and/or the like. A non-volatile computer-readable storage medium may also include a punch card, paper tape, optical mark sheet (or any other physical medium with patterns of holes or other optically recognizable indicia), compact disc read only memory (CD-ROM), compact disc-rewritable (CD-RW), digital versatile disc (DVD), Blu-ray disc (BD), any other non-transitory optical medium, and/or the like. Such a non-volatile computer-readable storage medium may also include read-only memory (ROM), programmable read-only memory (PROM), erasable programmable read-only memory (EPROM), electrically erasable programmable read-only memory (EEPROM), flash memory (e.g., Serial, NAND, NOR, and/or the like), multimedia memory cards (MMC), secure digital (SD) memory cards, SmartMedia cards, CompactFlash (CF) cards, Memory Sticks, and/or the like. Further, a non-volatile computer-readable storage medium may also include conductive-bridging random access memory (CBRAM), phase-change random access memory (PRAM), ferroelectric random-access memory (FeRAM), non-volatile random-access memory (NVRAM), magnetoresistive random-access memory (MRAM), resistive random-access memory (RRAM), Silicon-Oxide-Nitride-Oxide-Silicon memory (SONOS), floating junction gate random access memory (FJG RAM), Millipede memory, racetrack memory, and/or the like.

In one embodiment, a volatile computer-readable storage medium may include random access memory (RAM), dynamic random access memory (DRAM), static random access memory (SRAM), fast page mode dynamic random access memory (FPM DRAM), extended data-out dynamic random access memory (EDO DRAM), synchronous dynamic random access memory (SDRAM), double data rate synchronous dynamic random access memory (DDR SDRAM), double data rate type two synchronous dynamic random access memory (DDR2 SDRAM), double data rate type three synchronous dynamic random access memory (DDR3 SDRAM), Rambus dynamic random access memory (RDRAM), Twin Transistor RAM (TTRAM), Thyristor RAM (T-RAM), Zero-capacitor (Z-RAM), Rambus in-line memory module (RIMM), dual in-line memory module (DIMM), single in-line memory module (SIMM), video random access memory (VRAM), cache memory (including various levels), flash memory, register memory, and/or the like. It will be appreciated that where embodiments are described to use a computer-readable storage medium, other types of computer-readable storage media may be substituted for or used in addition to the computer-readable storage media described above.

As should be appreciated, various embodiments of the present invention may also be implemented as methods, apparatus, systems, computing devices, computing entities, and/or the like. As such, embodiments of the present invention may take the form of an apparatus, system, computing device, computing entity, and/or the like executing instructions stored on a computer-readable storage medium to perform certain steps or operations. Thus, embodiments of the present invention may also take the form of an entirely hardware embodiment, an entirely computer program product embodiment, and/or an embodiment that comprises combination of computer program products and hardware performing certain steps or operations. Embodiments of the present invention are described below with reference to block diagrams and flowchart illustrations. Thus, it should be understood that each block of the block diagrams and flowchart illustrations may be implemented in the form of a computer program product, an entirely hardware embodiment, a combination of hardware and computer program products, and/or apparatus, systems, computing devices, computing entities, and/or the like carrying out instructions, operations, steps, and similar words used interchangeably (e.g., the executable instructions, instructions for execution, program code, and/or the like) on a computer-readable storage medium for execution. For example, retrieval, loading, and execution of code may be performed sequentially such that one instruction is retrieved, loaded, and executed at a time. In some exemplary embodiments, retrieval, loading, and/or execution may be performed in parallel such that multiple instructions are retrieved, loaded, and/or executed together. Thus, such embodiments can produce specifically-configured machines performing the steps or operations specified in the block diagrams and flowchart illustrations. Accordingly, the block diagrams and flowchart illustrations support various combinations of embodiments for performing the specified instructions, operations, or steps.

### IV. Exemplary System Architecture

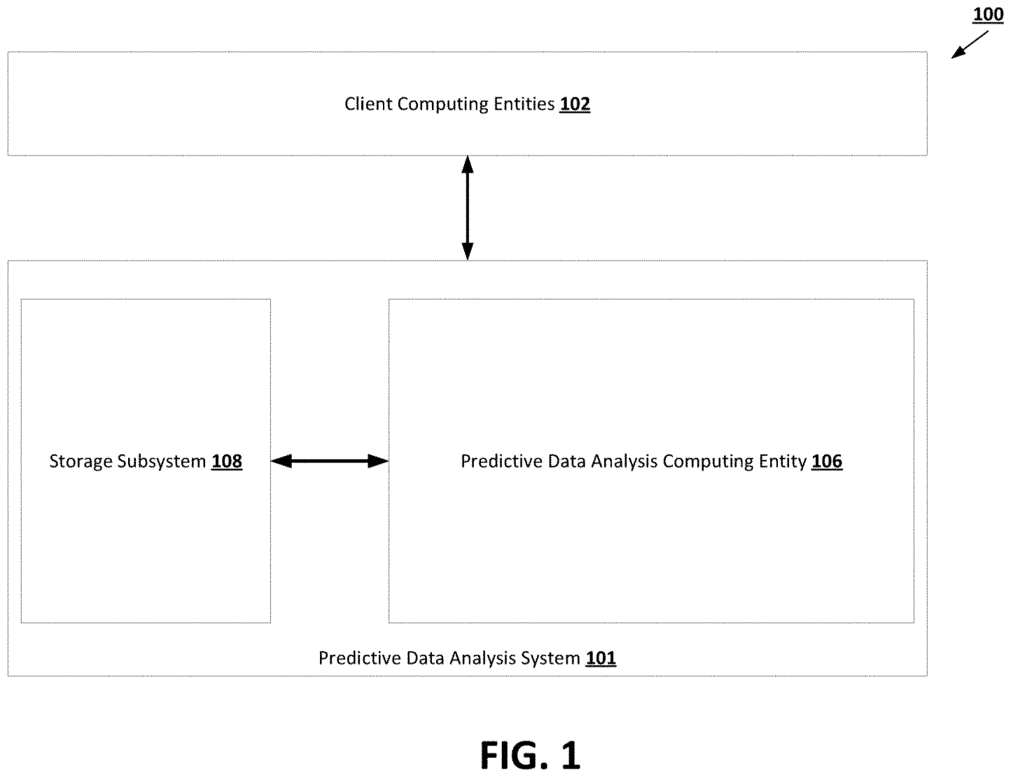

FIG. 1 is a schematic diagram of an example architecture 100 for performing predictive data analysis. The architecture 100 includes a predictive data analysis system 101 configured to receive predictive data analysis requests from client computing entities 102, process the predictive data analysis requests to generate predictions, provide the generated predictions to the client computing entities 102, and automatically perform prediction-based actions based at least in part on the generated predictions. An example of a prediction-based action that can be performed using the predictive data analysis system 101 is generating a set of recommended health actions for an individual. Another example of a prediction-based action that can be performed using the predictive data analysis system 101 is generating an overall predicted utility score for an individual.

In some embodiments, predictive data analysis system 101 may communicate with at least one of the client computing entities 102 using one or more communication networks. Examples of communication networks include any wired or wireless communication network including, for example, a wired or wireless local area network (LAN), personal area network (PAN), metropolitan area network (MAN), wide area network (WAN), or the like, as well as any hardware, software and/or firmware required to implement it (such as, e.g., network routers, and/or the like).

The predictive data analysis system 101 may include a predictive data analysis computing entity 106 and a storage subsystem 108. The predictive data analysis computing entity 106 may be configured to receive predictive data analysis requests from one or more client computing entities 102, process the predictive data analysis requests to generate predictions corresponding to the predictive data analysis requests, provide the generated predictions to the client computing entities 102, and automatically perform prediction-based actions based at least in part on the generated predictions.

The storage subsystem 108 may be configured to store input data used by the predictive data analysis computing entity 106 to perform predictive data analysis as well as model definition data used by the predictive data analysis computing entity 106 to perform various predictive data analysis tasks. The storage subsystem 108 may include one or more storage units, such as multiple distributed storage units that are connected through a computer network. Each storage unit in the storage subsystem 108 may store at least one of one or more data assets and/or one or more data about the computed properties of one or more data assets. Moreover, each storage unit in the storage subsystem 108 may include one or more non-volatile storage or memory media including, but not limited to, hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like.

### Exemplary Predictive Data Analysis Computing Entity

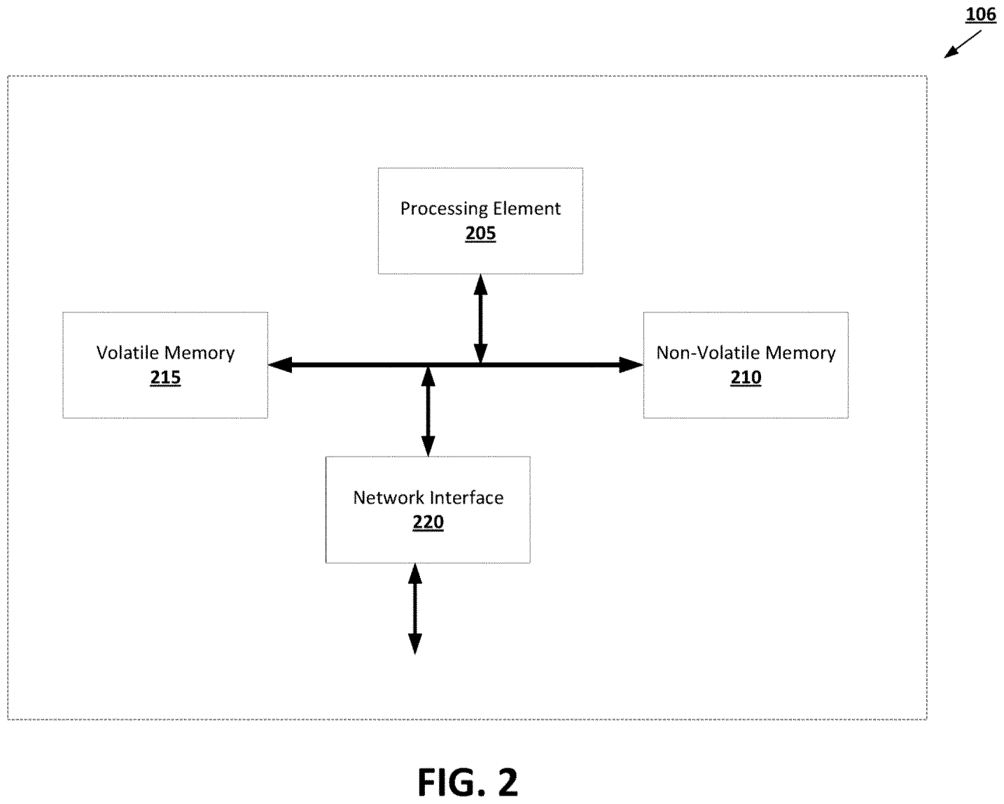

FIG. 2 provides a schematic of a predictive data analysis computing entity 106 according to one embodiment of the present invention. In general, the terms computing entity, computer, entity, device, system, and/or similar words used herein interchangeably may refer to, for example, one or more computers, computing entities, desktops, mobile phones, tablets, phablets, notebooks, laptops, distributed systems, kiosks, input terminals, servers or server networks, blades, gateways, switches, processing devices, processing entities, set-top boxes, relays, routers, network access points, base stations, the like, and/or any combination of devices or entities adapted to perform the functions, operations, and/or processes described herein. Such functions, operations, and/or processes may include, for example, transmitting, receiving, operating on, processing, displaying, storing, determining, creating/generating, monitoring, evaluating, comparing, and/or similar terms used herein interchangeably. In one embodiment, these functions, operations, and/or processes can be performed on data, content, information, and/or similar terms used herein interchangeably.

As indicated, in one embodiment, the predictive data analysis computing entity 106 may also include one or more communications interfaces 220 for communicating with various computing entities, such as by communicating data, content, information, and/or similar terms used herein interchangeably that can be transmitted, received, operated on, processed, displayed, stored, and/or the like.

As shown in FIG. 2, in one embodiment, the predictive data analysis computing entity 106 may include, or be in communication with, one or more processing elements 205 (also referred to as processors, processing circuitry, and/or similar terms used herein interchangeably) that communicate with other elements within the predictive data analysis computing entity 106 via a bus, for example. As will be understood, the processing element 205 may be embodied in a number of different ways.

For example, the processing element 205 may be embodied as one or more complex programmable logic devices (CPLDs), microprocessors, multi-core processors, coprocessing entities, application-specific instruction-set processors (ASIPs), microcontrollers, and/or controllers. Further, the processing element 205 may be embodied as one or more other processing devices or circuitry. The term circuitry may refer to an entirely hardware embodiment or a combination of hardware and computer program products. Thus, the processing element 205 may be embodied as integrated circuits, application specific integrated circuits (ASICs), field programmable gate arrays (FPGAs), programmable logic arrays (PLAs), hardware accelerators, other circuitry, and/or the like.

As will therefore be understood, the processing element 205 may be configured for a particular use or configured to execute instructions stored in volatile or non-volatile media or otherwise accessible to the processing element 205. As such, whether configured by hardware or computer program products, or by a combination thereof, the processing element 205 may be capable of performing steps or operations according to embodiments of the present invention when configured accordingly.

In one embodiment, the predictive data analysis computing entity 106 may further include, or be in communication with, non-volatile media (also referred to as non-volatile storage, memory, memory storage, memory circuitry and/or similar terms used herein interchangeably). In one embodiment, the non-volatile storage or memory may include one or more non-volatile storage or memory media 210, including, but not limited to, hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like.

As will be recognized, the non-volatile storage or memory media may store databases, database instances, database management systems, data, applications, programs, program modules, scripts, source code, object code, byte code, compiled code, interpreted code, machine code, executable instructions, and/or the like. The term database, database instance, database management system, and/or similar terms used herein interchangeably may refer to a collection of records or data that is stored in a computer-readable storage medium using one or more database models, such as a hierarchical database model, network model, relational model, entity-relationship model, object model, document model, semantic model, graph model, and/or the like.

In one embodiment, the predictive data analysis computing entity 106 may further include, or be in communication with, volatile media (also referred to as volatile storage, memory, memory storage, memory circuitry and/or similar terms used herein interchangeably). In one embodiment, the volatile storage or memory may also include one or more volatile storage or memory media 215, including, but not limited to, RAM, DRAM, SRAM, FPM DRAM, EDO DRAM, SDRAM, DDR SDRAM, DDR2 SDRAM, DDR3 SDRAM, RDRAM, TTRAM, T-RAM, Z-RAM, RIMM, DIMM, SIMM, VRAM, cache memory, register memory, and/or the like.

As will be recognized, the volatile storage or memory media may be used to store at least portions of the databases, database instances, database management systems, data, applications, programs, program modules, scripts, source code, object code, byte code, compiled code, interpreted code, machine code, executable instructions, and/or the like being executed by, for example, the processing element 205. Thus, the databases, database instances, database management systems, data, applications, programs, program modules, scripts, source code, object code, byte code, compiled code, interpreted code, machine code, executable instructions, and/or the like may be used to control certain aspects of the operation of the predictive data analysis computing entity 106 with the assistance of the processing element 205 and operating system.

As indicated, in one embodiment, the predictive data analysis computing entity 106 may also include one or more communications interfaces 220 for communicating with various computing entities, such as by communicating data, content, information, and/or similar terms used herein interchangeably that can be transmitted, received, operated on, processed, displayed, stored, and/or the like. Such communication may be executed using a wired data transmission protocol, such as fiber distributed data interface (FDDI), digital subscriber line (DSL), Ethernet, asynchronous transfer mode (ATM), frame relay, data over cable service interface specification (DOCSIS), or any other wired transmission protocol. Similarly, the predictive data analysis computing entity 106 may be configured to communicate via wireless external communication networks using any of a variety of protocols, such as general packet radio service (GPRS), Universal Mobile Telecommunications System (UMTS), Code Division Multiple Access 2000 (CDMA2000), CDMA2000 1× (1×RTT), Wideband Code Division Multiple Access (WCDMA), Global System for Mobile Communications (GSM), Enhanced Data rates for GSM Evolution (EDGE), Time Division-Synchronous Code Division Multiple Access (TD-SCDMA), Long Term Evolution (LTE), Evolved Universal Terrestrial Radio Access Network (E-UTRAN), Evolution-Data Optimized (EVDO), High Speed Packet Access (HSPA), High-Speed Downlink Packet Access (HSDPA), IEEE 802.11 (Wi-Fi), Wi-Fi Direct, 802.16 (WiMAX), ultra-wideband (UWB), infrared (IR) protocols, near field communication (NFC) protocols, Wibree, Bluetooth protocols, wireless universal serial bus (USB) protocols, and/or any other wireless protocol.

Although not shown, the predictive data analysis computing entity 106 may include, or be in communication with, one or more input elements, such as a keyboard input, a mouse input, a touch screen/display input, motion input, movement input, audio input, pointing device input, joystick input, keypad input, and/or the like. The predictive data analysis computing entity 106 may also include, or be in communication with, one or more output elements (not shown), such as audio output, video output, screen/display output, motion output, movement output, and/or the like.

### Exemplary Client Computing Entity

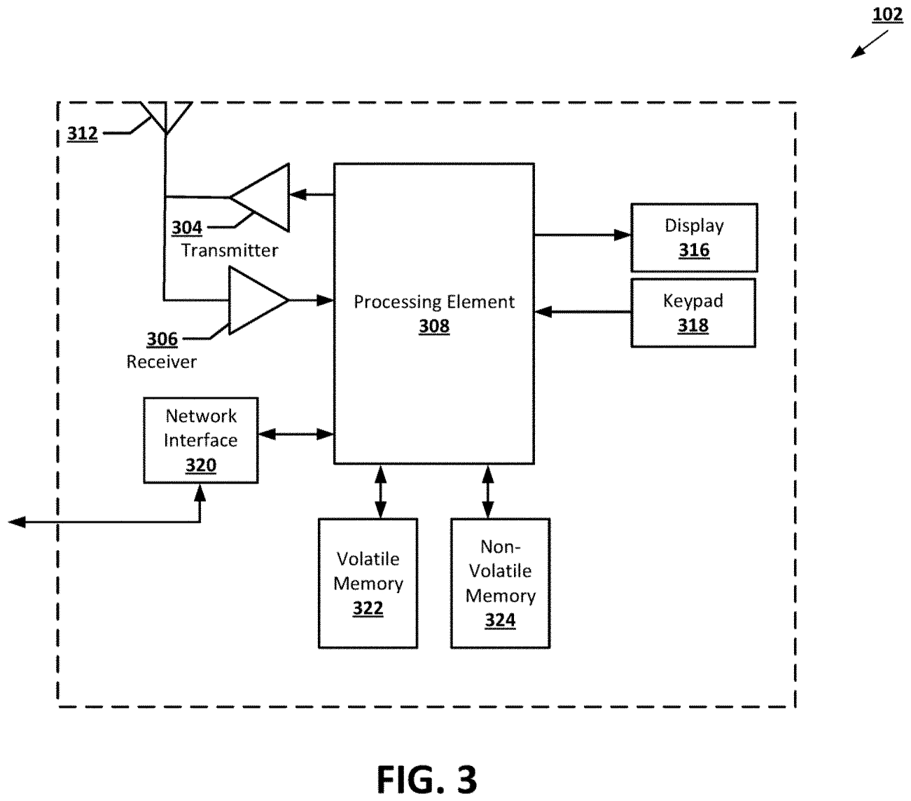

FIG. 3 provides an illustrative schematic representative of a client computing entity 102 that can be used in conjunction with embodiments of the present invention. In general, the terms device, system, computing entity, entity, and/or similar words used herein interchangeably may refer to, for example, one or more computers, computing entities, desktops, mobile phones, tablets, phablets, notebooks, laptops, distributed systems, kiosks, input terminals, servers or server networks, blades, gateways, switches, processing devices, processing entities, set-top boxes, relays, routers, network access points, base stations, the like, and/or any combination of devices or entities adapted to perform the functions, operations, and/or processes described herein. Client computing entities 102 can be operated by various parties. As shown in FIG. 3, the client computing entity 102 can include an antenna 312, a transmitter 304 (e.g., radio), a receiver 306 (e.g., radio), and a processing element 308 (e.g., CPLDs, microprocessors, multi-core processors, coprocessing entities, ASIPs, microcontrollers, and/or controllers) that provides signals to and receives signals from the transmitter 304 and receiver 306, correspondingly.

The signals provided to and received from the transmitter 304 and the receiver 306, correspondingly, may include signaling information/data in accordance with air interface standards of applicable wireless systems. In this regard, the client computing entity 102 may be capable of operating with one or more air interface standards, communication protocols, modulation types, and access types. More particularly, the client computing entity 102 may operate in accordance with any of a number of wireless communication standards and protocols, such as those described above with regard to the predictive data analysis computing entity 106. In a particular embodiment, the client computing entity 102 may operate in accordance with multiple wireless communication standards and protocols, such as UMTS, CDMA2000, 1×RTT, WCDMA, GSM, EDGE, TD-SCDMA, LTE, E-UTRAN, EVDO, HSPA, HSDPA, Wi-Fi, Wi-Fi Direct, WiMAX, UWB, IR, NFC, Bluetooth, USB, and/or the like. Similarly, the client computing entity 102 may operate in accordance with multiple wired communication standards and protocols, such as those described above with regard to the predictive data analysis computing entity 106 via a network interface 320.

Via these communication standards and protocols, the client computing entity 102 can communicate with various other entities using concepts such as Unstructured Supplementary Service Data (USSD), Short Message Service (SMS), Multimedia Messaging Service (MMS), Dual-Tone Multi-Frequency Signaling (DTMF), and/or Subscriber Identity Module Dialer (SIM dialer). The client computing entity 102 can also download changes, add-ons, and updates, for instance, to its firmware, software (e.g., including executable instructions, applications, program modules), and operating system.

According to one embodiment, the client computing entity 102 may include location determining aspects, devices, modules, functionalities, and/or similar words used herein interchangeably. For example, the client computing entity 102 may include outdoor positioning aspects, such as a location module adapted to acquire, for example, latitude, longitude, altitude, geocode, course, direction, heading, speed, universal time (UTC), date, and/or various other information/data. In one embodiment, the location module can acquire data, sometimes known as ephemeris data, by identifying the number of satellites in view and the relative positions of those satellites (e.g., using global positioning systems (GPS)). The satellites may be a variety of different satellites, including Low Earth Orbit (LEO) satellite systems, Department of Defense (DOD) satellite systems, the European Union Galileo positioning systems, the Chinese Compass navigation systems, Indian Regional Navigational satellite systems, and/or the like. This data can be collected using a variety of coordinate systems, such as the Decimal Degrees (DD); Degrees, Minutes, Seconds (DMS); Universal Transverse Mercator (UTM); Universal Polar Stereographic (UPS) coordinate systems; and/or the like. Alternatively, the location information/data can be determined by triangulating the client computing entity's 102 position in connection with a variety of other systems, including cellular towers, Wi-Fi access points, and/or the like. Similarly, the client computing entity 102 may include indoor positioning aspects, such as a location module adapted to acquire, for example, latitude, longitude, altitude, geocode, course, direction, heading, speed, time, date, and/or various other information/data. Some of the indoor systems may use various position or location technologies including RFID tags, indoor beacons or transmitters, Wi-Fi access points, cellular towers, nearby computing devices (e.g., smartphones, laptops) and/or the like. For instance, such technologies may include the iBeacons, Gimbal proximity beacons, Bluetooth Low Energy (BLE) transmitters, NFC transmitters, and/or the like. These indoor positioning aspects can be used in a variety of settings to determine the location of someone or something to within inches or centimeters.

The client computing entity 102 may also comprise a user interface (that can include a display 316 coupled to a processing element 308) and/or a user input interface (coupled to a processing element 308). For example, the user interface may be a user application, browser, user interface, and/or similar words used herein interchangeably executing on and/or accessible via the client computing entity 102 to interact with and/or cause display of information/data from the predictive data analysis computing entity 106, as described herein. The user input interface can comprise any of a number of devices or interfaces allowing the client computing entity 102 to receive data, such as a keypad 318 (hard or soft), a touch display, voice/speech or motion interfaces, or other input device. In embodiments including a keypad 318, the keypad 318 can include (or cause display of) the conventional numeric (0-9) and related keys (#, *), and other keys used for operating the client computing entity 102 and may include a full set of alphabetic keys or set of keys that may be activated to provide a full set of alphanumeric keys. In addition to providing input, the user input interface can be used, for example, to activate or deactivate certain functions, such as screen savers and/or sleep modes.

The client computing entity 102 can also include volatile storage or memory 322 and/or non-volatile storage or memory 324, which can be embedded and/or may be removable. For example, the non-volatile memory may be ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like. The volatile memory may be RAM, DRAM, SRAM, FPM DRAM, EDO DRAM, SDRAM, DDR SDRAM, DDR2 SDRAM, DDR3 SDRAM, RDRAM, TTRAM, T-RAM, Z-RAM, RIMM, DIMM, SIMM, VRAM, cache memory, register memory, and/or the like. The volatile and non-volatile storage or memory can store databases, database instances, database management systems, data, applications, programs, program modules, scripts, source code, object code, byte code, compiled code, interpreted code, machine code, executable instructions, and/or the like to implement the functions of the client computing entity 102. As indicated, this may include a user application that is resident on the entity or accessible through a browser or other user interface for communicating with the predictive data analysis computing entity 106 and/or various other computing entities.

In another embodiment, the client computing entity 102 may include one or more components or functionality that are the same or similar to those of the predictive data analysis computing entity 106, as described in greater detail above. As will be recognized, these architectures and descriptions are provided for exemplary purposes only and are not limiting to the various embodiments.

In various embodiments, the client computing entity 102 may be embodied as an artificial intelligence (AI) computing entity, such as an Amazon Echo, Amazon Echo Dot, Amazon Show, Google Home, and/or the like. Accordingly, the client computing entity 102 may be configured to provide and/or receive information/data from a user via an input/output mechanism, such as a display, a camera, a speaker, a voice-activated input, and/or the like. In certain embodiments, an AI computing entity may comprise one or more predefined and executable program algorithms stored within an onboard memory storage module, and/or accessible over a network. In various embodiments, the AI computing entity may be configured to retrieve and/or execute one or more of the predefined program algorithms upon the occurrence of a predefined trigger event.

### V. Exemplary System Operations

Various embodiments of the present invention improve the efficiency of performing utility evaluation by introducing techniques to determine predicted utility scores by mapping values from a risk domain to a utility domain. The inventors have concluded that, in a lot of application contexts, risk scores are more readily available than utility scores. By generating predicted utility scores by mapping risk scores to utility scores, various embodiments of the present invention avoid the need for performing computationally intensive operations to generate utility measures from raw feature data. In this way, various embodiments of the present invention improve the computational efficiency of performing utility evaluation.

Moreover, various embodiments of the present invention improve the operational reliability of predicted utility scores generated using performing utility evaluation by generating predicted utility scores based at least in part on a relationship between a utility distribution measure value for the utility discretization category that is associated with an observed utility discretization category and each utility distribution measure value for a plurality of utility discretization categories. In some embodiments, the noted task may be performed based at least in part on the output of the equation

$$\frac{u - min}{max - min} ,$$

where min is the minimum value of the utility distribution values for the set of utility discretization categories and max is the maximum value of the utility distribution values for the set of utility discretization categories. In some embodiments, the operations of the described equation (and potentially other normalization equations) have the effect that, if u=max, the predicted utility score is one, and if u=min, the predicted utility score is zero, a technique which enhances the diversity of the predicted utility scores within an allowable range and does not “waste” any unused subranges of the allowable range. In this way, the noted embodiments of the present invention generate predicted utility scores that have a wider precision and thus are deemed more predictively informative and operationally reliable. Accordingly, by utilizing the described techniques, various embodiments of the present invention improve the operational reliability of predicted utility scores generated using performing utility evaluation.

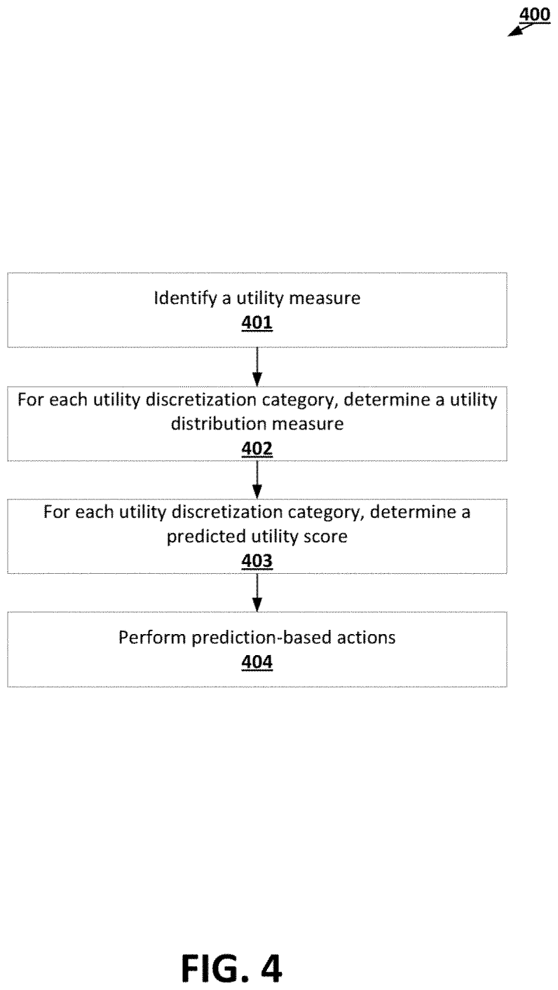

FIG. 4 is a flowchart diagram of an example process 400 for predictive utility evaluation for a utility measure value (e.g., of a monitored entity, such as a monitored individual) given a defined demographic profile. Via the various steps/operations of the process 400, a predictive data analysis computing entity 106 can determine a predicted utility score for a monitored entity (e.g., a monitored individual) in a manner that takes into account both a defined demographic profile of the monitored entity and a utility measure value describing an observed condition state and/or an activity state of the monitored entity.

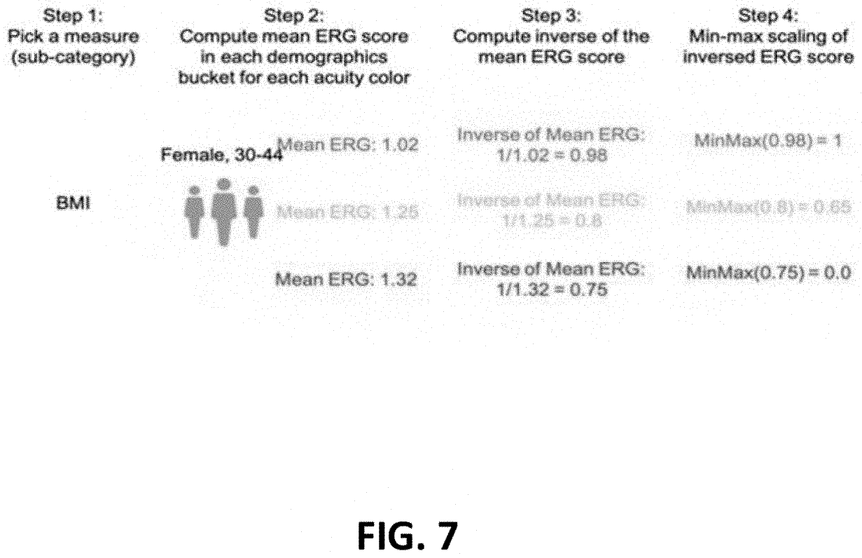

The process 400 begins at step/operation 401 when the predictive data analysis computing entity 106 identifies a utility measure for the utility measure value. An operational example of performing step/operation 401 is depicted in FIG. 7. As depicted in FIG. 7, a utility measure related to BMI biometric measurements is determined for a defined demographic profile of females having 30 to 44 years of age. While the exemplary embodiment of FIG. 7 uses mean Episode Risk Group (ERG) as a risk distribution measure, a person of ordinary skill in the relevant technology will recognize that any statistical distribution measure for ERG or for a non-ERG risk measure may be used.

In some embodiments, the utility measure is a utility sub-category (e.g., a health sub-category) of a utility category (e.g., a health category) that is associated with a monitored entity (e.g., a monitored individual). Examples of utility categories include a biometrics utility category, a diet utility category, an exercise utility category, a sleep utility category, and a state of mind utility category. Examples of utility sub-categories for a biometrics utility category include a BMI utility sub-category, a dyslipidemia utility sub-category, a blood pressure utility sub-category, and/or the like. Examples of utility sub-categories for a nutrition utility category include a nutrition score utility sub-category, a calorie count utility sub-category, and/or the like. Examples of utility sub-categories for an exercise utility category include an aerobic exercise session count utility sub-category, an exercise session count utility sub-category, a weight-bearing exercise session count utility sub-category, and/or the like. Examples of utility sub-categories for a sleep utility category include a sleep hour magnitude utility sub-category, a sleep quality score utility sub-category, and/or the like. Examples of utility sub-categories for a state of mind utility category include a mood score utility sub-category, a social isolation score utility sub-category, a stress score utility sub-category, and/or the like. In some embodiments, a utility category describes a biometrics utility category and a utility sub-category for the utility category describes a particular biometric measurement. In some embodiments, a utility category describes an exercise activity utility category and a utility sub-category of the utility category describes a recorded count of performance of a particular exercise activity.

In some embodiments, a utility measure describes describe an aspect of an overall utility measurement with respect to an observed set of phenomena. For example, a set of utility measures may include a set of measurements/observations that relate to health state and/or health activities of a monitored individual. In some embodiments, a utility measure may be associated with a utility category and a utility sub-category. For example, a utility measure that describes an aspect of a health state and/or a health activity of a monitored individual may have a utility category that describes that the utility measure pertains to biometric observations/measurements, and a utility sub-category that describes that the utility measure pertains to a particular biometric observation/measurement, such as to a BMI biometric observation/measurement. In some embodiments, a utility category describes an exercise activity utility category and a utility sub-category of the utility category describes a recorded count of performance of a particular exercise activity. In some embodiments, a utility measure value is a particular value of a utility measure that may be associated with a monitored entity (e.g., a monitored individual). For example, if a utility measure is associated with weekly exercise session count, then a utility measure value may describe a weekly exercise session count of 4 that may be associated with a particular monitored individual.

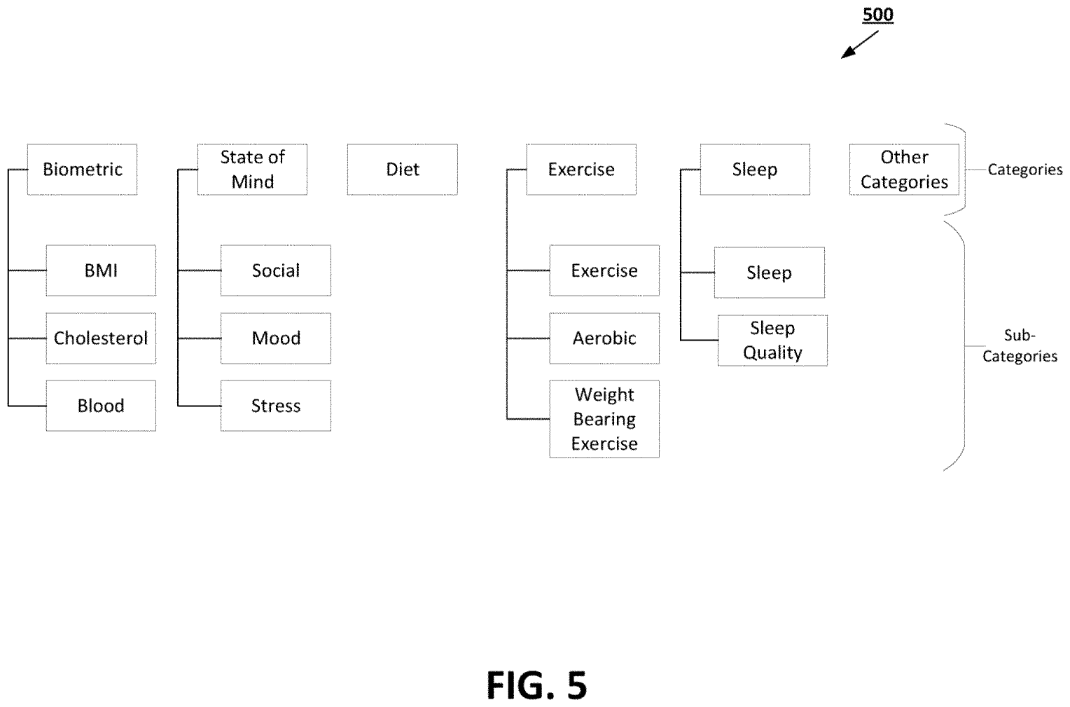

An operational example of a utility measure hierarchical scheme 500 from which a utility measure may be selected is depicted in FIG. 5. As depicted in FIG. 5 the utility measure hierarchical scheme 500 includes a set of utility categories (including biometric, state of mind, diet, exercise, and sleep) and a set of utility sub-categories. As further depicted in FIG. 5, each utility sub-category is associated with one and only one utility category. In some embodiments, selecting a utility measure comprises traversing a utility measure hierarchical scheme such as the utility measure hierarchical scheme 500 of FIG. 5.

At step/operation 402, the predictive data analysis computing entity 106 determines a utility distribution measure value for each utility discretization category that is associated with the utility measure value. For example, in some embodiments, the predictive data analysis computing entity 106 computes a mean of all ERG scores for all monitored individuals that are associated with the defined demographic profile and whose utility measure values fall within the set of utility measure values for a utility discretization category, and then determines each utility distribution measure value for a utility discretization category based at least in part on the computed mean for the utility discretization category. An operational example of performing step/operation 402 is depicted in FIG. 7. As depicted in FIG. 7, step/operation 402 includes generating a utility distribution measure designated as “Inverse of Mean ERG” for each utility discretization category of three utility discretization categories.

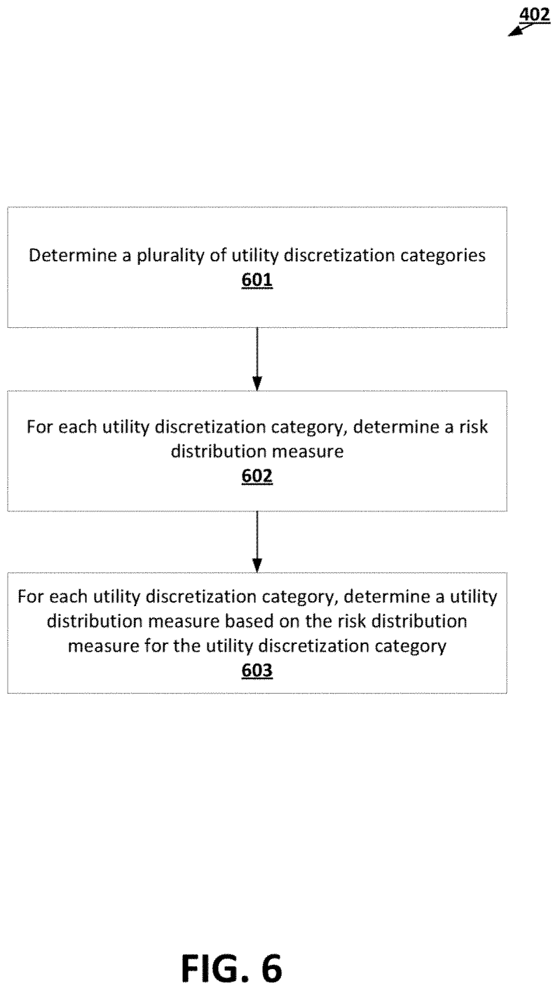

In some embodiments, step/operation 402 may be performed in accordance with the process that is depicted in FIG. 6. The process that is depicted in FIG. 6 begins at step/operation 601 when the predictive data analysis computing entity 106 determines a plurality of (e.g., a defined number of) utility discretization categories for the utility measure value. In some embodiments, each utility discretization category is defined bucket/sub-range of potential utility measure values for a utility measure.

In some embodiments, a utility discretization category is associated with a set of utility measure values associated with a utility measure. In some embodiments, all of the potential utility measure values for a particular utility measures are divided into a set of disjoint utility discretization categories, such that each potential utility measure value is assigned to not more than and no less than one utility discretization category. For example, if a utility measure is a continuous value (e.g., a BMI measure), then a total range of the utility measure may be divided into a number of sub-ranges each corresponding to a utility discretization category. In one example, if a utility measure may have a continuous value in the range [0, 1], then the utility discretization categories for the utility measure may include a first utility discretization category for utility measure values that fall in the sub-range [0, 0.25), a second utility discretization category for utility measure values that fall in the sub-range [0.25, 0.5), a third utility discretization category for utility measure values that fall in the sub-range [0.5, 0.75), and a fourth utility discretization category for utility measure values that fall in the sub-range [0.75, 1]. As another example, if a utility measure is a discrete value (e.g., a number of weekly exercise sessions), then all potential discrete values for the utility measure may be divided into a number of “buckets” each corresponding to a utility discretization category. In one example, if a utility measure may have a discrete value from the set {0, 1, 2, 3, 4}, then the utility discretization categories for the utility measure may include a first utility discretization category for utility measure values that fall in the subset {0, 1}, a third utility discretization category for utility measure values that fall in the subset {2, 3}, and a third utility discretization category for utility measure values that fall in the subset {4}.

At step/operation 602, the predictive data analysis computing entity 106 determines a risk distribution measure value for each utility discretization category. For example, in some embodiments, the predictive data analysis computing entity 106 computes a mean of all ERG scores for all monitored individuals that are associated with the defined demographic profile and whose utility measure values fall within the set of utility measure values for a utility discretization category, and then determines each risk distribution measure value for a utility discretization category based at least in part on the computed mean for the utility discretization category. However, while various embodiments of the present invention describe using mean values to determine risk distribution measure values, a person of ordinary skill in the relevant technology will recognize that other statistical distribution values, such as other centroid statistical distribution values, can be used to determine risk distribution measures values. Other examples of statistical distribution values that can be used to determine risk distribution measures values include median values, weighted mean values, mode values, and/or the like. In some embodiments, each risk distribution measure value describes a centroid statistical distribution measure for all risk measure values (e.g., ERG values) for monitored entities (e.g., monitored individuals) that are associated with the defined demographic profile and the utility discretization category for the risk distribution measure value.

In some embodiments, a risk distribution measure value is a measure of statistical distribution of a risk measure across a defined demographic profile given a utility discretization category that is associated with the risk distribution measure. For example, consider a utility discretization category that describes performing three or four weekly exercises and a defined demographic profile of women between 40-50 years of age that live in the United States and have diabetes. In the noted example, the risk distribution measure value may describe a statistical distribution measure (e.g., a mean) of a risk measure (e.g., an episode risk group (ERG) measure) across all monitored individuals that correspond to the defined demographic profile (i.e., across all monitored individuals that are women between 40-50 years of age, live in the United States, and have diabetes). Thus, in the noted example, each utility measure such as a weekly exercise session count utility measure may be associated with n risk distribution measure values, where n is the number of utility discretization categories (e.g., buckets) associated with the utility measure. In some embodiments, to determine a risk distribution measure value for a defined demographic profile given a utility discretization category, the following operations may be performed: (i) identifying all of the monitored entities (e.g., all of the monitored individuals) that both satisfy the defined demographic profile (e.g., are associated with the required demographic features) and are associated with utility measure values that fall within the set of utility measures values associated with the utility discretization category (e.g., that have been recorded to have exercised the required number of times weekly); (ii) for each identified monitored entity, determining a relevant risk measure value (e.g., an ERG value); (iii) compute a measure of statistical distribution of each relevant risk measure value for an identified monitored entity; and (iv) determine the risk distribution measure value based at least in part on the measure of statistical distribution.

At step/operation 603, the predictive data analysis computing entity 106 determines a utility distribution measure value for each utility discretization category based at least in part on the risk discretization measure for the utility discretization category. In some embodiments, the predictive data analysis computing entity 106 determines a utility distribution measure value for each utility discretization category based at least in part on inverting the risk discretization measure for the utility discretization category. In some embodiments, the predictive data analysis computing entity 106 determines a utility distribution measure value for each utility discretization category based at least in part on negating the risk discretization measure for the utility discretization category. In some embodiments, a utility distribution measure value is determined based at least in part on the output of a transformation of a risk distribution measure value to a utility domain.

In some embodiments, a risk distribution measure value is associated with a risk domain (e.g., the ERG risk domain), which may describe a scheme for mapping a set of monitored entities into a set of numbers/values (e.g., a set of risk distribution measure values) such that a monitored entity deemed to be associated with greater risk is associated with a higher number/value in the risk domain. Therefore, if monitored entity m₁ is deemed to be in a riskier condition than the monitored entity m₂ (e.g., m₁ is deemed to have a BMI that is further away from a healthy BMI range than the BMI for m₂), then the mapped number/value for m₁ in the risk domain is higher than the mapped number/value for m₂ in the risk domain.

On the other hand, a utility distribution measure value is associated with a utility domain, which may describe a scheme for mapping a set of monitored entities into a set of numbers/values (e.g., a set of utility distribution measure values) such that a monitored entity deemed to be associated with greater risk is associated with a lower number/value in the utility domain. Therefore, if monitored entity m₁ is deemed to be in a riskier condition than the monitored entity m₂ (e.g., m₁ is deemed to have a BMI that is further away from a healthy BMI range than the BMI for m₂), then the mapped number/value for m₁ in the utility domain is lower than the mapped number/value for m₂ in the utility domain. Thus, in some embodiments, generating a utility distribution measure value for a utility discretization category comprises identifying the risk distribution measure value for the utility discretization category and then mapping the risk distribution measure value into the utility domain in order to generate the utility distribution measure value for the utility discretization category. This may include, in some embodiments, inversing or negating the risk distribution measure value for the particular utility discretization category. For example, in some embodiments, given a utility discretization category that is associated with the risk distribution measure value r, the utility discretization measure value for the utility discretization category is determined based at least in part on the output of 1/r or r⁻¹.

In some embodiments, by using the techniques of the step/operation 603, various embodiments of the present invention improve the efficiency of performing utility evaluation by introducing techniques to determine predicted utility scores by mapping values from a risk domain to a utility domain. The inventors have concluded that, in a lot of application contexts, risk scores are more readily available than utility scores. By generating predicted utility scores by mapping risk scores to utility scores, various embodiments of the present invention avoid the need for performing computationally intensive operations to generate utility measures from raw feature data. In this way, various embodiments of the present invention improve the computational efficiency of performing utility evaluation.

Returning to FIG. 4, at step/operation 403, the predictive data analysis computing entity 106 determines a predicted utility score for each utility discretization category based at least in part on a relationship between the utility distribution measure value for the utility discretization category and each utility distribution measure value for the plurality of utility discretization categories. An operational example of performing step/operation 403 is depicted in FIG. 7. As depicted in FIG. 7, step/operation 403 includes generating a predicted utility score (designated as the MinMax(x) where x is the described utility distribution measure value for a corresponding utility discretization category) for each utility discretization category of three utility discretization categories.

In some embodiments, step/operation 403 includes determining the predicted utility score for a particular utility measure value that is associated with an observed utility discretization category of a plurality of utility discretization categories, where the noted sub-step/sub-operation may be performed based at least in part on a relationship between the utility distribution measure value for the utility discretization category that is associated with observed utility discretization category and each utility distribution measure value for the plurality of utility discretization categories. In some embodiments, step/operation 403 includes determining the predicted utility score for a particular monitored entity whose utility measure value is associated with an observed utility discretization category of a plurality of utility discretization categories, where the noted sub-step/sub-operation may be performed based at least in part on a relationship between the utility distribution measure value for the utility discretization category that is associated with observed utility discretization category and each utility distribution measure value for the plurality of utility discretization categories.

In some embodiments, a predicted utility score describes, for a utility measure value (e.g., of a monitored entity such as a monitored individual) that is associated with a particular observed utility discretization category, a relationship between the utility distribution measure value for the observed utility discretization category and each utility distribution measure value for the plurality of utility discretization categories. In some embodiments, a predicted utility score describes, for a particular observed utility discretization category, a relationship between the utility distribution measure value for the observed utility discretization category and each utility distribution measure value for the plurality of utility discretization categories.

In some embodiments, each utility measure value is associated with a particular utility discretization category, and each utility discretization category is associated with a utility distribution measure. For example, a particular utility measure that is associated with weekly exercise session counts may be associated with a first utility discretization category having a utility distribution measure of u₁ that applies to monitored entities having {0, 1} weekly exercise session counts, a second utility discretization category having a utility distribution measure u₂ that applies to monitored entities having {2, 3, 4} weekly exercise session counts, and a third utility discretization category having a utility distribution measure u₃ that applies to monitored individuals having more than 4 weekly exercise session counts. In the noted example, the predicted utility score for a monitored individual having three weekly exercise session counts may be determined based at least in part on a relationship between u₂ and {u₁, u₂, u₃}. For example, the predicted utility score may be determined based at least in part on normalizing (e.g., min-max normalization of) each of u₁, u₂, and u₃. As another example, the predicted utility score may be determined based at least in part on: the utility distribution measure value for the observed utility discretization category (e.g., u₂), a minimal predicted utility score for the plurality of utility discretization categories (a minimum value of u₁, u₂, and u₃), and a maximal predicted utility score for the plurality of utility discretization categories (a maximum value of u₁, u₂, and u₃). In some embodiments, given a utility distribution measure value of u for a particular monitored individual that is associated with a particular utility discretization category of a set of utility discretization categories, the predicted utility score for the particular monitored individual and/or for the particular utility discretization category may be determined based at least in part on the output of the equation

$$\frac{u - min}{max - min} ,$$

where min is the minimum value of the utility distribution values for the set of utility discretization categories and max is the maximum value of the utility distribution values for the set of utility discretization categories. In some embodiments, the operations of the described equation (and potentially other normalization equations) have the effect that, if u=max, the predicted utility score is one, and if u=min, the predicted utility score is zero, a technique which enhances the diversity of the predicted utility scores within an allowable range and does not “waste” any unused subranges of the allowable range.

In some embodiments, by using the techniques of the step/operation 403, various embodiments of the present invention improve the operational reliability of predicted utility scores generated using performing utility evaluation by generating predicted utility scores based at least in part on a relationship between a utility distribution measure value for the utility discretization category that is associated with an observed utility discretization category and each utility distribution measure value for a plurality of utility discretization categories. In some embodiments, the noted task may be performed based at least in part on the output of the equation

$$\frac{u - min}{max - min} ,$$

where min is the minimum value of the utility distribution values for the set of utility discretization categories and max is the maximum value of the utility distribution values for the set of utility discretization categories. In some embodiments, the operations of the described equation (and potentially other normalization equations) have the effect that, if u=max, the predicted utility score is one, and if u=min, the predicted utility score is zero, a technique which enhances the diversity of the predicted utility scores within an allowable range and does not “waste” any unused subranges of the allowable range. In this way, the noted embodiments of the present invention generate predicted utility scores that have a wider precision and thus are deemed more predictively informative and operationally reliable. Accordingly, by utilizing the described techniques, various embodiments of the present invention improve the operational reliability of predicted utility scores generated using performing utility evaluation.

At step/operation 404, the predictive data analysis computing entity 106 performs one or more prediction-based actions based at least in part on the predicted utility measure. In some embodiments, performing the one or more prediction-based actions is performed based at least in part on an overall utility measure for a monitored entity based at least in part on each utility measure value for the monitored entity. Examples of prediction-based actions comprise generating automated notifications, generating automated alerts, performing load balancing actions, automated action scheduling, automated appointment scheduling, automated drug presentation, and/or the like.

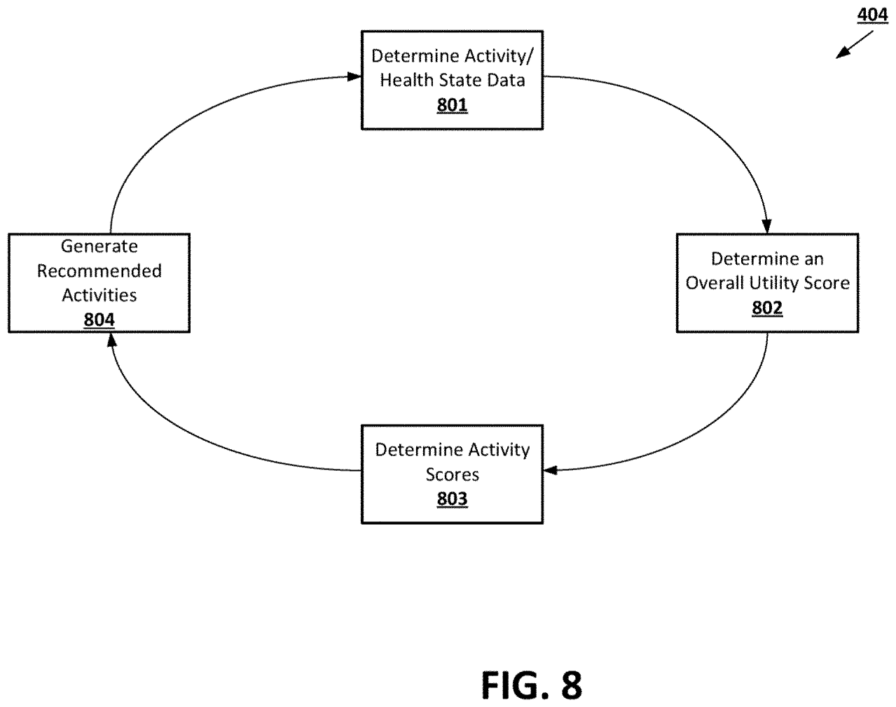

In some embodiments, step/operation 404 may be performed in accordance with the process that is depicted in FIG. 8. The process that is depicted in FIG. 8 starts at step/operation 801 when the predictive data analysis computing entity 106 determines activity/health state data for a monitored entity. Examples of activity/health state data include health survey data, biometric data (e.g., biometric data originating from one or more biometric sensors), medical/health claim data, user device data, user web interaction data, user interaction data with one or more software applications, user demographic data, user social interaction data, user social media interaction data, user preference data, and/or the like.

At step/operation 802, the predictive data analysis computing entity 106 determines an overall utility score for a monitored entity that is associated with the utility measure value based at least in part on the activity/health state data. In some embodiments, each monitored entity is associated with a set of utility measure values for a set of utility measures, where each utility measure is in turn associated with a utility category and a utility sub-category. In some embodiments, an overall utility score describes a combination of all utility measure values for a monitored entity across all utility categories.

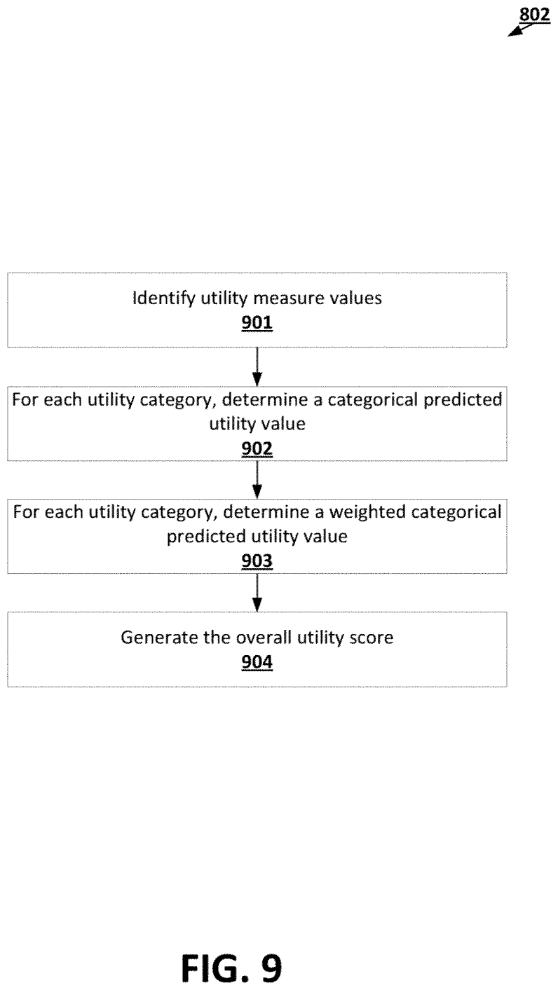

In some embodiments, step/operation 802 may be performed in accordance with the process that is depicted in FIG. 9. The process that is depicted in FIG. 9 begins at step/operation 901 when the predictive data analysis computing entity 106 identifies a set of utility measure values for a monitored entity. As discussed above, each utility measure may in in turn be associated with a utility category and a utility sub-category. In some embodiments, an overall utility score describes a combination of all utility measure values for a monitored entity across all utility categories. Examples of utility categories include a biometrics utility category, a diet utility category, an exercise utility category, a sleep utility category, and a state of mind utility category. Examples of utility sub-categories for a biometrics utility category include a BMI utility sub-category, a dyslipidemia utility sub-category, a blood pressure utility sub-category, and/or the like. Examples of utility sub-categories for a nutrition utility category include a nutrition score utility sub-category, a calorie count utility sub-category, and/or the like. Examples of utility sub-categories for an exercise utility category include an aerobic exercise session count utility sub-category, an exercise session count utility sub-category, a weight-bearing exercise session count utility sub-category, and/or the like. Examples of utility sub-categories for a sleep utility category include a sleep hour magnitude utility sub-category, a sleep quality score utility sub-category, and/or the like. Examples of utility sub-categories for a state of mind utility category include a mood score utility sub-category, a social isolation score utility sub-category, a stress score utility sub-category, and/or the like.

At step/operation 902, the predictive data analysis computing entity 106 determines a categorical predicted utility value for each utility category by combining each predicted utility score for a utility measure value that is associated with the utility category (i.e., for each utility sub-category that is associated with the utility category). For example, if utility sub-categories for a biometrics utility category include a BMI utility sub-category, a dyslipidemia utility sub-category, and a blood pressure utility sub-category, the predictive data analysis computing entity 106 may generate the categorical utility value for the biometrics utility category by combining (e.g., summing up, multiplying, computing a mean of, a computing a median of, and/or the like) the predicted utility value for the BMI utility sub-category, the predicted utility value for the dyslipidemia utility sub-category, and the predicted utility value for the blood pressure utility sub-category.

At step/operation 903, the predictive data analysis computing entity 106 determines a weighted categorical predicted utility value for each utility category based at least in part on the categorical predicted utility value for the utility category and a categorical utility weight for the utility category. In some embodiments, to determine the weighted categorical predicted utility value for a utility category, the predictive data analysis computing entity 106 applies the categorical utility weight for the utility category to the categorical predicted utility value for the utility category. The categorical utility weight for a utility category may be a predefined static value, a trained static value, or a dynamically generated value. In some embodiments, the categorical utility weight for a utility category describes an estimated significance of a utility category to an overall utility measurement for a monitored entity.

At step/operation 904, the predictive data analysis computing entity 106 combines each weighted categorical predicted utility value for a utility category of the plurality of utility categories to determine the overall predicted utility value for the monitored entity. In some embodiments, to determine the overall predicted utility value for the monitored entity, the predictive data analysis computing entity 106 computes a mean of each weighted categorical predicted utility value for a utility category of the plurality of utility categories. In some embodiments, to determine the overall predicted utility value for the monitored entity, the predictive data analysis computing entity 106 computes a sum of each weighted categorical predicted utility value for a utility category of the plurality of utility categories. In some embodiments, to determine the overall predicted utility value for the monitored entity, the predictive data analysis computing entity 106 computes a median of each weighted categorical predicted utility value for a utility category of the plurality of utility categories.

Returning to FIG. 8, at step/operation 803, the predictive data analysis computing entity 106 determines an activity score for each activity of a set of candidate activities based at least in part on the overall utility score (e.g., based at least in part on an overall health score). In some embodiments, an activity score for a candidate activity describes an estimated/computed recommendation value for a monitored entity performing the candidate activity given the overall utility score for the candidate activity.

At step/operation 804, the predictive data analysis computing entity 106 generates recommended activities for the monitored entity based at least in part on activity scores for the candidate activities. In some embodiments, the predictive data analysis computing entity 106 recommends performing any candidate activity whose activity score satisfies an activity score threshold. In some embodiments, the predictive data analysis computing entity 106 recommends performing any activity that is among the top n activities with top n activity scores, where n may be a predefined, tuned, and/or trained hyper-parameter of the predictive data analysis system 101. In some embodiments, each candidate activity is associated with a candidate activity type of one or more candidate activity types. In some embodiments, for each candidate activity type, the predictive data analysis computing entity 106 recommends performing any activity that is among the top m activities that are associated with the candidate activity type with top m activity scores among the activities that are associated with the candidate activity, where m may be a predefined, tuned, and/or trained hyper-parameter of the predictive data analysis system 101. In some embodiments, at step/operation 804, the predictive data analysis computing entity 106 generates automated notifications and/or alerts for recommended activities. In some embodiments, at step/operation 804, the predictive data analysis computing entity 106 generates user interface data for a prediction output user interface that may be presented to an end user by the predictive data analysis computing entity 106 and/or by a client computing entity 102, where the prediction output user interface may describe the recommended activities and/or explanatory metadata describing why/how the recommended activities are generated based at least in part on overall predicted utility scores and/or activity scores.

Steps/operations 801-804 may be repeated over time to detect new activities by a monitored entity, where the detected activity data may be used to generate new overall predicted utility scores, new activity scores, and new recommended activities. In some embodiments, steps/operations 801-804 may be repeated over time until terminated by a stopping condition. In some embodiments, steps/operations 801-804 may be repeated over time until terminated by a stopping event. In some embodiments, steps/operations 801-804 may be repeated over time until s number of iterations, where s may be a predefined, tuned, and/or trained hyper-parameter of the predictive data analysis system 101.

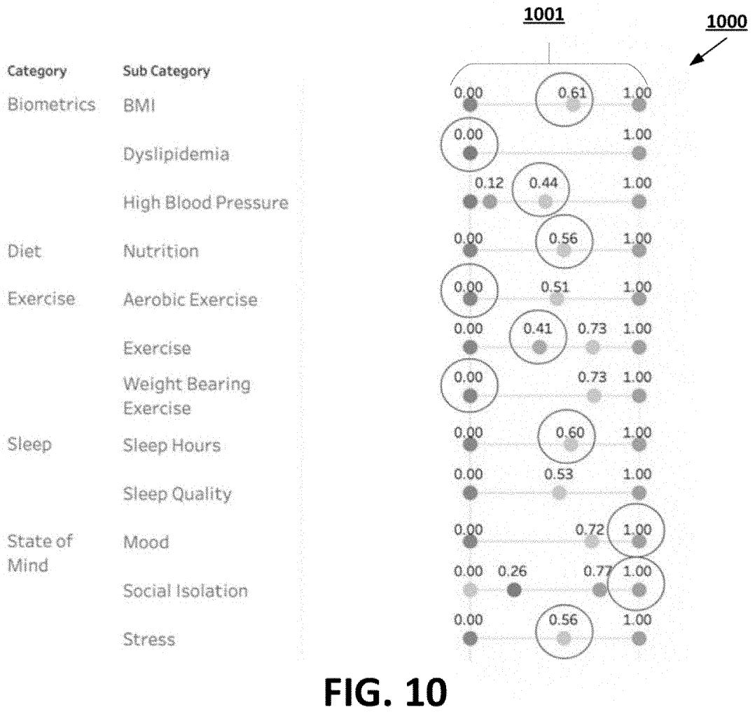

In some embodiments, performing the prediction output user interface comprises generating user interface data for a prediction output user interface describing a relationship between the predicted utility score and a distribution of predicted utility scores for the plurality of utility discretization categories. An operational example of this prediction output user interface 1000 is depicted in FIG. 10. As depicted in FIG. 10, the prediction output user interface describes, for each utility measure value, a measure of relationship between the predicted utility scores for the utility measure value and a distribution of predicted utility scores for the plurality of utility discretization categories. For example, for the utility measure value that is associated with a BMI utility measure for a particular individual, the prediction output user interface 1000 includes the user interface element 1001, which describes the relationship between the predicted utility score for the BMI utility measure value (i.e., the predicted utility score of 0.61) and a distribution of predicted utility scores for the plurality of utility discretization categories (i.e., a distribution characterized in part by a maximal predicted utility score of 1.0 and a minimal predicted utility score of 0.0). In some embodiments, the prediction output user interface 1000 may be presented to an end user by the predictive data analysis computing entity 106 and/or by a client computing entity 102.

Thus, as described above, various embodiments of the present invention improve the efficiency of performing utility evaluation by introducing techniques to determine predicted utility scores by mapping values from a risk domain to a utility domain. The inventors have concluded that, in a lot of application contexts, risk scores are more readily available than utility scores. By generating predicted utility scores by mapping risk scores to utility scores, various embodiments of the present invention avoid the need for performing computationally intensive operations to generate utility measures from raw feature data. In this way, various embodiments of the present invention improve the computational efficiency of performing utility evaluation.

Moreover, as further described above. various embodiments of the present invention improve the operational reliability of predicted utility scores generated using performing utility evaluation by generating predicted utility scores based at least in part on a relationship between a utility distribution measure value for the utility discretization category that is associated with an observed utility discretization category and each utility distribution measure value for a plurality of utility discretization categories. In some embodiments, the noted task may be performed based at least in part on the output of the equation

$$\frac{u - min}{max - min} ,$$

where min is the minimum value of the utility distribution values for the set of utility discretization categories and max is the maximum value of the utility distribution values for the set of utility discretization categories. In some embodiments, the operations of the described equation (and potentially other normalization equations) have the effect that, if u=max, the predicted utility score is one, and if u=min, the predicted utility score is zero, a technique which enhances the diversity of the predicted utility scores within an allowable range and does not “waste” any unused subranges of the allowable range. In this way, the noted embodiments of the present invention generate predicted utility scores that have a wider precision and thus are deemed more predictively informative and operationally reliable. Accordingly, by utilizing the described techniques, various embodiments of the present invention improve the operational reliability of predicted utility scores generated using performing utility evaluation.

### VI. Conclusion

Many modifications and other embodiments will come to mind to one skilled in the art to which this disclosure pertains having the benefit of the teachings presented in the foregoing descriptions and the associated drawings. Therefore, it is to be understood that the disclosure is not to be limited to the specific embodiments disclosed and that modifications and other embodiments are intended to be included within the scope of the appended claims. Although specific terms are employed herein, they are used in a generic and descriptive sense only and not for purposes of limitation.

1. A computer-implemented method for predictive utility evaluation for a utility measure value given a defined demographic profile, the computer-implemented method comprising:
identifying, by a processor, a plurality of utility discretization categories for a utility measure that is associated with the utility measure value, wherein: (i) each utility discretization category is associated with a risk distribution measure value for the defined demographic profile, and (ii) the plurality of utility discretization categories comprise an observed utility discretization category that describes a current utility measure value for the utility measure value;
for each utility discretization category, by the processor, determining a utility distribution measure value based at least in part on transforming the risk distribution measure value for the corresponding utility discretization category into a utility domain;
determining, by the processor, the predicted utility score based at least in part on a relationship between the utility distribution measure value for the utility discretization category that is associated with the observed utility discretization category and each utility distribution measure value for the plurality of utility discretization categories; and
performing, by the processor, one or more prediction-based actions based at least in part on the predicted utility score.

2. The computer-implemented method of claim 1, wherein each risk distribution measure value for a particular utility discretization category describes a centroid statistical distribution measure for all risk measure values for monitored entities that are associated with the defined demographic profile and the particular utility discretization category.

3. The computer-implemented method of claim 2, wherein the risk measure is an episode risk group (ERG) measure.

4. The computer-implemented method of claim 1, wherein the utility measure is associated with a utility category and a utility sub-category.

5. The computer-implemented method of claim 4, wherein the utility category describes a biometrics utility category and the utility sub-category describes a particular biometric measurement.

6. The computer-implemented method of claim 4, wherein the utility category describes an exercise activity utility category and the utility sub-category describes a recorded count of performance of a particular exercise activity.

7. The computer-implemented method of claim 1, wherein performing the prediction-based actions comprises:
generating user interface data describing a relationship between the predicted utility score and a distribution of predicted utility scores for the plurality of utility discretization categories.

8. The computer-implemented method of claim 1, wherein the utility distribution measure value for a particular utility discretization category is determined based at least in part on an inverse of the risk distribution measure value for the particular utility discretization category.

9. The computer-implemented method of claim 1, wherein the predicted utility score is determined based at least in part on:
the utility distribution measure value for the observed utility discretization category,
a minimal predicted utility score for the plurality of utility discretization categories, and
a maximal predicted utility score for the plurality of utility discretization categories.

10. The computer-implemented method of claim 1, wherein performing the one or more prediction-based actions comprises:
combining one or more common-category predicted utility scores for one or more utility measure values of a monitored individual that is associated with the utility measure value to generate a categorical utility measure value for a particular utility category of a plurality of utility categories that is associated with the utility measure value, wherein: (i) the one or more utility measure values are associated with one or more utility measures that are all associated with the particular utility category, (ii) the one or more utility measure values comprise the utility measure value, (iii) the one or more utility measures comprise the utility measure for the utility measure value, and (iv) the one or more common-category predicted utility scores comprise the predicted utility score for the utility measure value;
determining, for each utility category, a categorical utility weight;
determining an overall predicted utility score based at least in part on each predicted utility value for the plurality of utility categories and each categorical utility weight for the plurality of utility categories; and
performing the one or more prediction-based actions based at least in part on the overall predicted utility score.

11. An apparatus for predictive utility evaluation for a utility measure value given a defined demographic profile, the apparatus comprising at least one processor and at least one memory including program code, the at least one memory and the program code configured to, with the processor, cause the apparatus to at least:
identify a plurality of utility discretization categories for a utility measure that is associated with the utility measure value, wherein: (i) each utility discretization category is associated with a risk distribution measure value for the defined demographic profile, and (ii) the plurality of utility discretization categories comprise an observed utility discretization category that describes a current utility measure value for the utility measure value;
for each utility discretization category, determine a utility distribution measure value based at least in part on transforming the risk distribution measure value for the utility discretization category into a utility domain;
determine the predicted utility score based at least in part on a relationship between the utility distribution measure value for the utility discretization category that is associated with the observed utility discretization category and each utility distribution measure value for the plurality of utility discretization categories; and
perform one or more prediction-based actions based at least in part on the predicted utility score.

12. The apparatus of claim 11, wherein each risk distribution measure value for a particular utility discretization category describes a centroid statistical distribution measure for all risk measure values for monitored entities that are associated with the defined demographic profile and the particular utility discretization category.

13. The apparatus of claim 12, wherein the risk measure is an episode risk group (ERG) measure.

14. The apparatus of claim 11, wherein the utility measure is associated with a utility category and a utility sub-category.

15. The apparatus of claim 11, wherein performing the prediction-based actions comprises:
generating user interface data describing a relationship between the predicted utility score and a distribution of predicted utility scores for the plurality of utility discretization categories.

16. The apparatus of claim 11, wherein the utility distribution measure value for a particular utility discretization category is determined based at least in part on an inverse of the risk distribution measure value for the particular utility discretization category.

17. The apparatus of claim 11, wherein the predicted utility score is determined based at least in part on:
the utility distribution measure value for the observed utility discretization category,
a minimal predicted utility score for the plurality of utility discretization categories, and
a maximal predicted utility score for the plurality of utility discretization categories.

18. The apparatus of claim 11, wherein performing the one or more prediction-based actions comprises:
combining one or more common-category predicted utility scores for one or more utility measure values of a monitored individual that is associated with the utility measure value to generate a categorical utility measure value for a particular utility category of a plurality of utility categories that is associated with the utility measure value, wherein: (i) the one or more utility measure values are associated with one or more utility measures that are all associated with the particular utility category, (ii) the one or more utility measure values comprise the utility measure value, (iii) the one or more utility measures comprise the utility measure for the utility measure value, and (iv) the one or more common-category predicted utility scores comprise the predicted utility score for the utility measure value;
determining, for each utility category, a categorical utility weight;
determining an overall predicted utility score based at least in part on each predicted utility value for the plurality of utility categories and each categorical utility weight for the plurality of utility categories; and
performing the one or more prediction-based actions based at least in part on the overall predicted utility score.

19. A computer program product for predictive utility evaluation for a utility measure value given a defined demographic profile, the computer program product comprising at least one non-transitory computer-readable storage medium having computer-readable program code portions stored therein, the computer-readable program code portions configured to:
identify a plurality of utility discretization categories for a utility measure that is associated with the utility measure value, wherein: (i) each utility discretization category is associated with a risk distribution measure value for the defined demographic profile, and (ii) the plurality of utility discretization categories comprise an observed utility discretization category that describes a current utility measure value for the utility measure value;
for each utility discretization category, determine a utility distribution measure value based at least in part on transforming the risk distribution measure value for the utility discretization category into a utility domain;
determine the predicted utility score based at least in part on a relationship between the utility distribution measure value for the utility discretization category that is associated with the observed utility discretization category and each utility distribution measure value for the plurality of utility discretization categories; and
perform one or more prediction-based actions based at least in part on the predicted utility score.

20. The computer program product of claim 19, wherein performing the one or more prediction-based actions comprises:
combining one or more common-category predicted utility scores for one or more utility measure values of a monitored individual that is associated with the utility measure value to generate a categorical utility measure value for a particular utility category of a plurality of utility categories that is associated with the utility measure value, wherein: (i) the one or more utility measure values are associated with one or more utility measures that are all associated with the particular utility category, (ii) the one or more utility measure values comprise the utility measure value, (iii) the one or more utility measures comprise the utility measure for the utility measure value, and (iv) the one or more common-category predicted utility scores comprise the predicted utility score for the utility measure value;
determining, for each utility category, a categorical utility weight;
determining an overall predicted utility score based at least in part on each predicted utility value for the plurality of utility categories and each categorical utility weight for the plurality of utility categories; and
performing the one or more prediction-based actions based at least in part on the overall predicted utility score.
